(19) United States
(12) Patent Application Publication
(10) Pub. No.: US 2026/0102271 A1
(43) Pub. Date: Apr. 16, 2026

(54) APPARATUS FOR KNEE EXTENSION AND FLEXION

(71) Applicant: TS Ideal Products, LLC, Park City, UT (US)

(72) Inventor: Jeffrey Lawrence Sharpe, Park City, UT (US)

(21) Appl. No.: 19/354,428

(22) Filed: Oct. 9, 2025

Related U.S. Application Data
(60) Provisional application No. 63/705,669, filed on Oct. 10, 2024.

(51) Int. Cl. A61F 5/01

(52) U.S. Cl. CPC A61F 5/0125; A61F 2005/0155

(57) ABSTRACT

An apparatus for knee extension and flexion worn about a leg of a user. The apparatus includes a frame having a generally parallel first and second frame sides, a handle end and a foot support end. The first frame side includes a first upper leg portion, a first lower leg portion, and a first knee hinge. The second frame side includes a second upper leg portion, a second lower leg portion, and a second knee hinge. The apparatus includes a handle having a handle pivot end and a handle distal end and the handle configured to rotate the first lower leg portion relative to the first upper leg portion. The apparatus includes a knee angle sensor to measure an angle at the first knee hinge.

### CROSS-REFERENCE TO RELATED APPLICATIONS

This application relates to and claims the benefit of U.S. Provisional Application No. 63/705,669 filed October 10, 2024, and entitled “APPARATUS FOR KNEE EXTENSION AND FLEXION” the entire disclosure of which is hereby wholly incorporated by reference

### BACKGROUND

Knee braces worn about a leg of a user are often used in conjunction with knee injury or knee surgery recovery processes. Generally speaking, it is often a goal during the recovery process to allow the lower leg to be able to move forward-most as possible towards an orientation of the lower leg approaching parallel to the upper leg for full knee extension. This facilitates the ligaments, muscles and other structures associated with the knee to properly heal and achieve desired mobility. However, performing such knee extensions while the knee is still in the recovery process may pose risks of further injury of recovery setbacks if not done correctly with the proper alignment or too much extension. U.S. Patent No. 12,357,491, entitled APPARATUS FOR KNEE EXTENSION FOR USE WITH KNEE BRACE, to Sharpe, discloses an apparatus that includes a frame that attaches to a knee brace. The apparatus features a handle that can be pulled by the user to place the user’s knee in extension for therapeutic purposes. However, it is often a goal to place the knee in flexion (i.e., a bending of the knee joint and opposite of extension/straightening).

In view of the foregoing, there is a need in the art for an improved apparatus for knee extension and flexion activities.

### BRIEF SUMMARY

According to an aspect of the invention, there is provided an apparatus for knee extension and flexion worn about a leg of a user. The apparatus includes a frame defining a generally elongated rectangular-like frame having a generally parallel first and second frame sides, a frame handle end and a foot support end bent towards the frame handle end. The first frame side includes a first upper leg portion, a first lower leg portion, and a first knee hinge disposed between and connecting the first upper leg portion and the first lower leg portion. The first knee hinge is sized and configured to allow the first lower leg portion to rotate relative to the first upper leg portion. The second frame side includes a second upper leg portion, a second lower leg portion, and a second knee hinge disposed between and connecting the second upper leg portion and the second lower leg portion. The second knee hinge is sized and configured to allow the second lower leg portion to rotate relative to the second upper leg portion. The apparatus further includes a handle having a handle pivot end and a handle distal end. The handle pivot end is connected to the frame handle end. The handle is sized and configured to allow the handle to pivot relative to the frame. The handle is disposed in mechanical communication with the first lower leg portion and sized and configured to rotate the first lower leg portion relative to the first upper leg portion upon pivoting of the handle relative to the frame. The apparatus further includes a knee angle sensor sized and configured to measure an angle at the first knee hinge between the first upper leg portion and the first lower leg portion and electronically generate angle data related to the measured angle.

According to various embodiments, the apparatus may further include a ratchet mechanism connected to the handle. The ratchet mechanism is in mechanical communication with the first lower leg portion and sized and configured to rotate the first lower leg portion in a rotational direction relative to the first upper leg portion upon pivoting of the handle relative to the frame. The ratchet mechanism may be configured to selectively control direction of rotation of the first lower leg portion relative to the first upper leg portion upon pivoting of the handle relative to the frame. The apparatus may further include a handle sprocket attached to the ratchet mechanism, a hinge sprocket disposed attached to the first lower leg portion adjacent the first knee hinge, and a drive chain engaged with the handle sprocket and the hinge sprocket. The drive chain is sized and configured to rotate the hinge sprocket upon rotation of the handle sprocket. Rotation of the hinge sprocket rotates the first lower leg portion relative to the first upper leg portion. The handle may be connected to the first upper leg portion and the second upper leg portion at the frame handle end. The apparatus may further include a processor in electronic communication with the knee angle sensor and sized and configured to receive the angle data. The apparatus may further include a transmitter in electronic communication with the processor and sized and configured to transmit a signal related to the angle data. The apparatus may further include a force sensor sized and configured to sense torque at the knee hinge with respect to forces applied to the first lower leg portion and electronically generate force data related to the sensed torque. The apparatus may further include a processor in electronic communication with the force sensor and sized and configured to receive the force data, and the apparatus may further include a transmitter in electronic communication with the processor and sized and configured to transmit a signal related to the force data. The force sensor may be sized and configured to sense torque at the knee hinge with respect to forces applied to the first lower leg by sensing torque with respect to the ratchet mechanism. The apparatus may further include a foot support disposed adjacent the foot support end and spanning across the first and second frame sides. The foot support may be sized and configured to engage a bottom of a foot of the user being disposed against the foot support. The apparatus may further include a heel wheel disposed adjacent the foot support sized and configured to rotate upon rolling of the heel wheel along a surface for movement of the heel support. The foot support may be sized and configured to selectively change angular position relative to the first and second frame sides. The heel wheel may be sized and configured to selectively change rolling resistance of the heel wheel. The apparatus may further include a resistance sensor sized and configured to sense rolling resistance with respect to the heel wheel and electronically generate wheel resistance data related to the sensed rolling resistance. The apparatus may further include a processor in electronic communication with the resistance sensor and sized and configured to receive the resistance data. The apparatus may further include a transmitter in electronic communication with the processor and sized and configured to transmit a signal related to the resistance data.

According to another aspect of the invention, there is provided an apparatus for knee extension and flexion worn about a leg of a user. The apparatus includes a frame defining a generally elongated rectangular-like frame having a generally parallel first and second frame sides, a frame handle end and a foot support end bent towards the frame handle end. The first frame side includes a first upper leg portion, a first lower leg portion, and a first knee hinge disposed between and connecting the first upper leg portion and the first lower leg portion. The first knee hinge is sized and configured to allow the first lower leg portion to rotate relative to the first upper leg portion. The second frame side includes a second upper leg portion, a second lower leg portion, and a second knee hinge disposed between and connecting the second upper leg portion and the second lower leg portion. The second knee hinge is sized and configured to allow the second lower leg portion to rotate relative to the second upper leg portion. The apparatus further includes a handle having a handle pivot end and a handle distal end. The handle pivot end is connected to the frame handle end. The handle is sized and configured to allow the handle to pivot relative to the frame. The handle is disposed in mechanical communication with the first lower leg portion and sized and configured to rotate the first lower leg portion relative to the first upper leg portion upon pivoting of the handle relative to the frame. The apparatus further includes a force sensor sized and configured to sense torque at the knee hinge with respect to forces applied to the first lower leg portion and electronically generate force data related to the sensed torque.

According to various embodiments, the apparatus may further include a processor in electronic communication with the force sensor and sized and configured to receive the force data. The apparatus may further include a transmitter in electronic communication with the processor and sized and configured to transmit a signal related to the force data. The apparatus may further include a ratchet mechanism connected to the handle. The ratchet mechanism may be in mechanical communication with the first lower leg portion and sized and configured to rotate the first lower leg portion in a rotational direction relative to the first upper leg portion upon pivoting of the handle relative to the frame. The force sensor may be sized and configured to sense torque at the knee hinge with respect to forces applied to the first lower leg by sensing torque with respect to the ratchet mechanism. The apparatus may further include a knee angle sensor sized and configured to measure an angle at the first knee hinge between the first upper leg portion and the first lower leg portion and electronically generate angle data related to the measured angle.

The present invention will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective front view of a user as wearing an apparatus for knee extension and flexion according to an aspect of the present invention along with a symbolic schematic and a symbolic remote device with electronic components shown as dashed lined boxes and leg straps shown in dashed lining;

FIG. 2 is a perspective back view of the apparatus of FIG. 1;

FIG. 3 is a side view of the apparatus of FIG. 1 as depicted with a user’s leg in dashed lining and a handle in various positions in dashed lining;

FIG. 4 is the side view of the apparatus of FIG. 3 with a first lower leg portion of the apparatus in a different angular position with the handle additionally indicated in dashed lining in a different angular position;

FIG. 5 is the side view of the apparatus of FIG. 3 with a first lower leg portion of the apparatus in a different angular position with the first lower leg portion aligned with a first upper leg portion with the user’s leg in a straight configuration;

FIG. 6 is the side view of the apparatus of FIG. 5 as depicted with a roller wheel of the apparatus on a surface;

FIG. 7 is the side view of the apparatus of FIG. 6 with the first lower leg portion of the apparatus in a different angular position with the handle in a forward position

FIG. 8 is an exploded perspective front view of the apparatus of FIG. 1;

FIG. 9 is a front perspective view of the ratchet mechanism of the apparatus of FIG. 1;

FIG. 10 is an exploded front perspective view of the ratchet mechanism of FIG. 9;

FIG. 11 is an exploded back perspective view of the ratchet mechanism of FIG. 9; and

FIG. 12 is a front perspective view of the apparatus of FIG. 1 in a straight configuration.

### DETAILED DESCRIPTION

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments. It is further understood that the use of relational terms such as top and bottom, first and second, and the like are used solely to distinguish one entity from another without necessarily requiring or implying any actual such relationship or order between such entities.

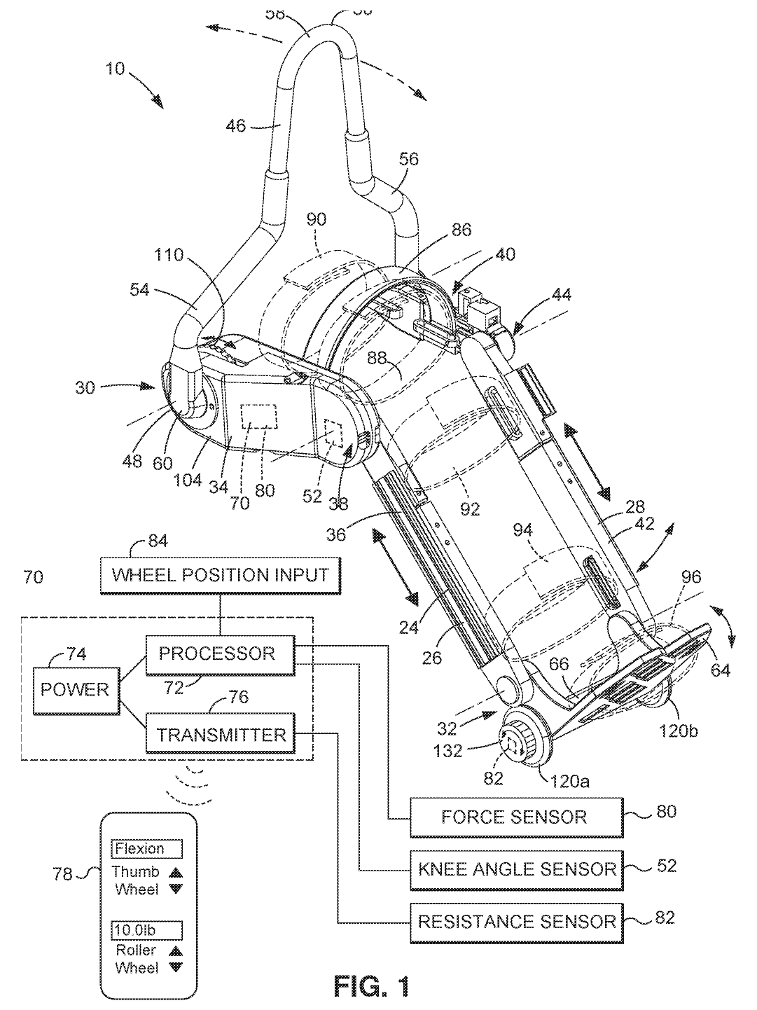

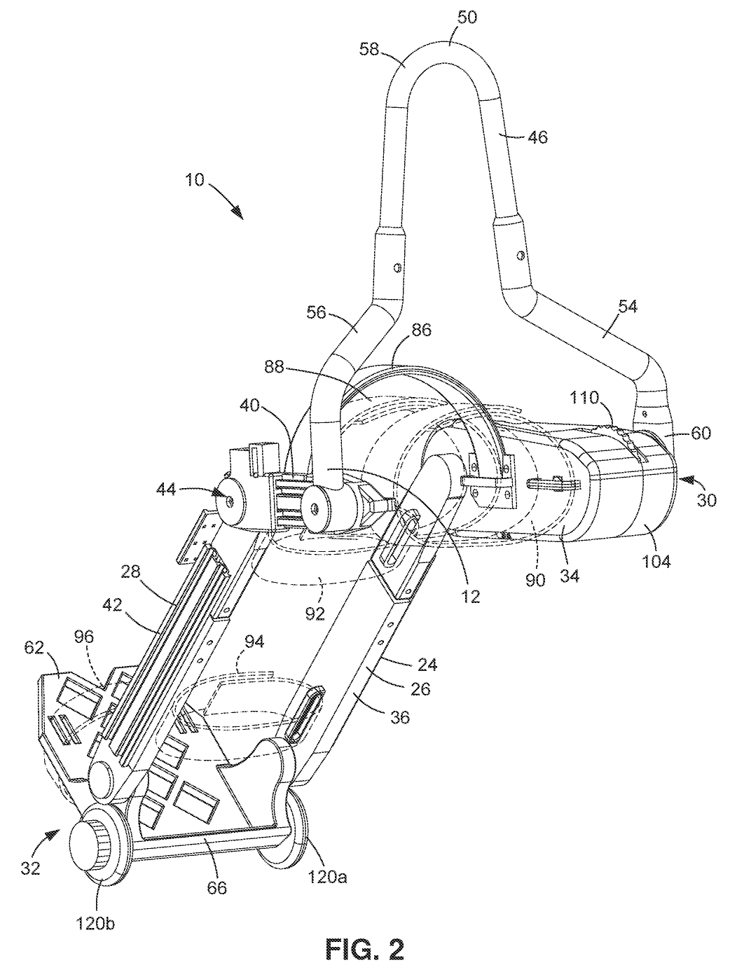

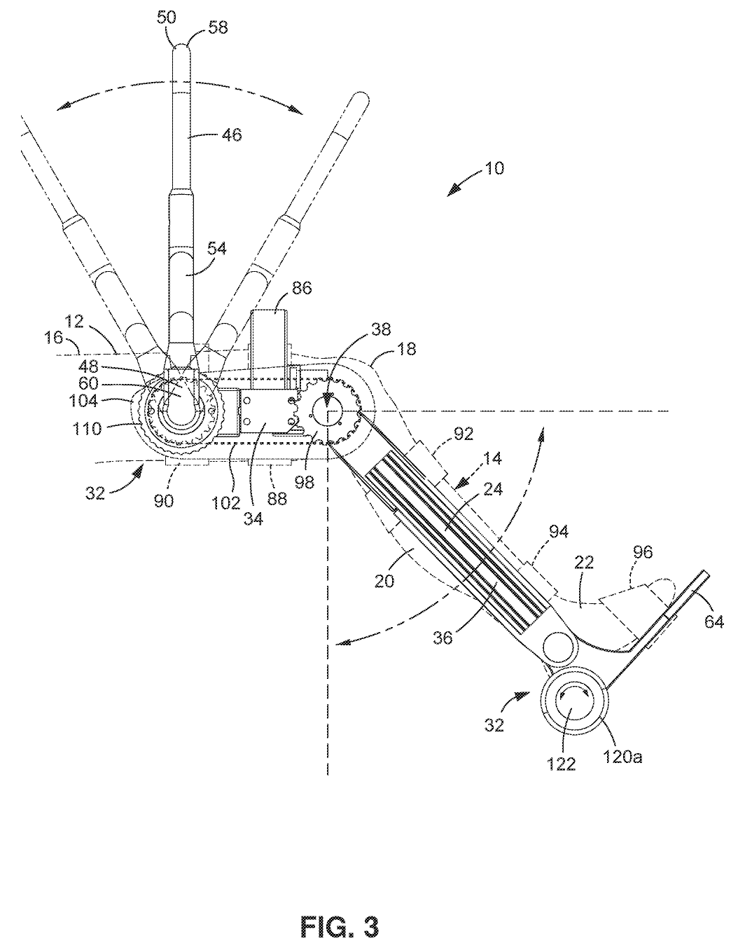

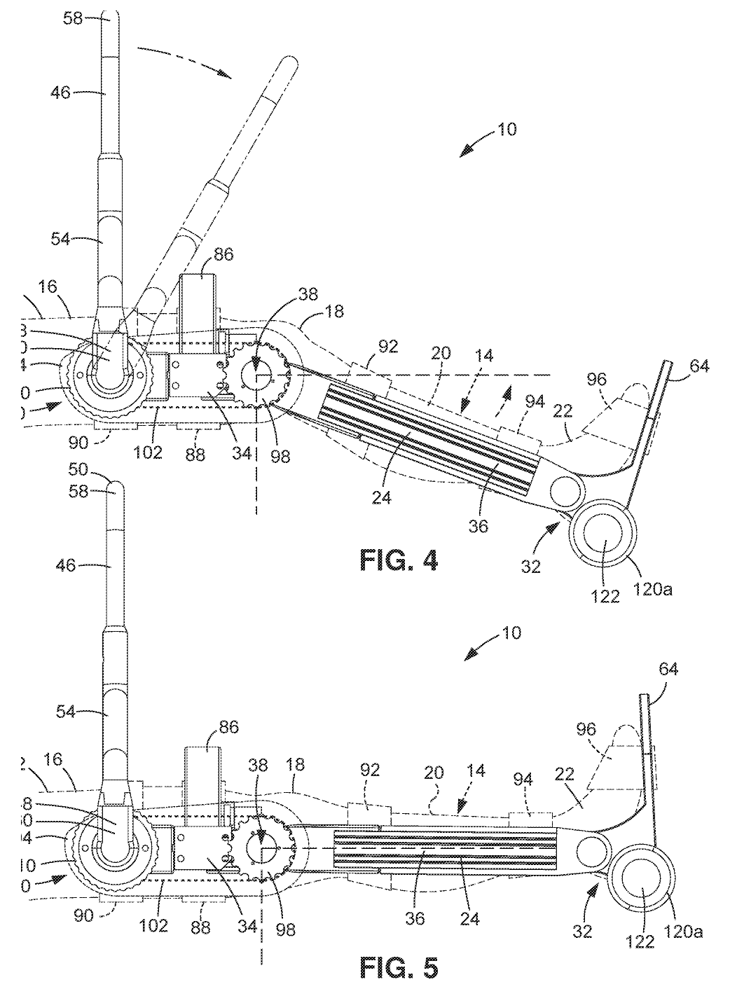

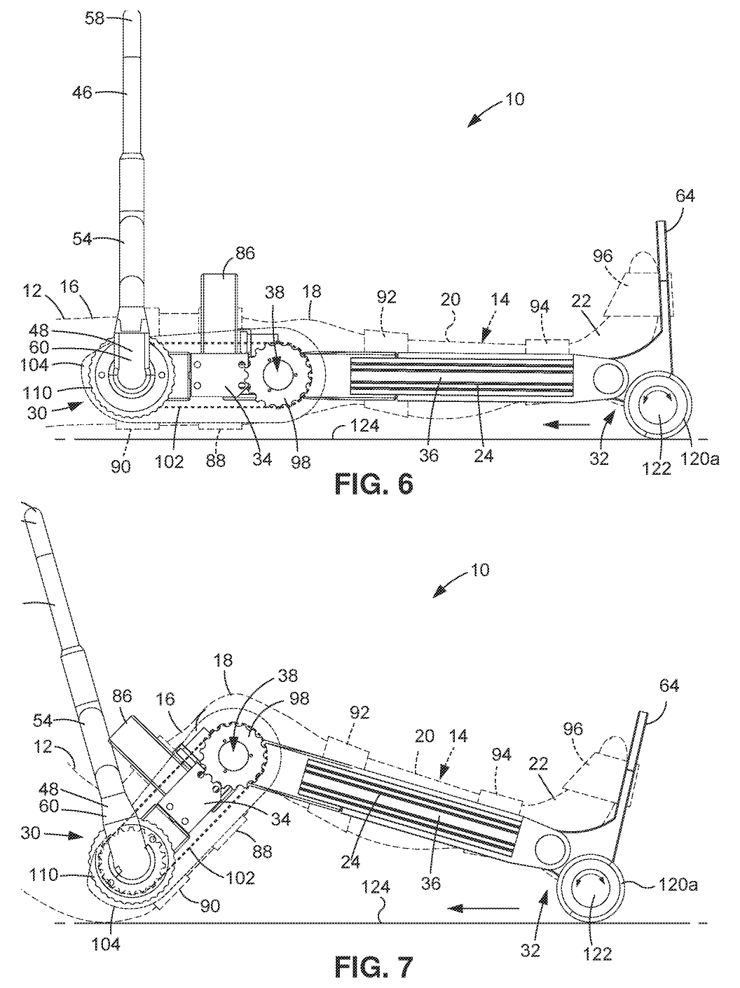

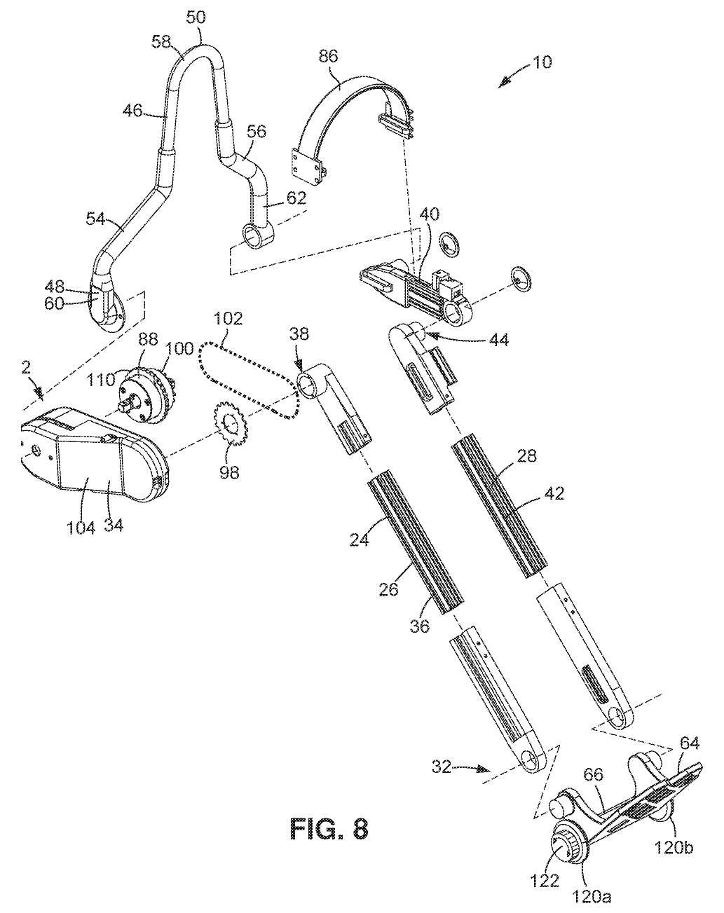

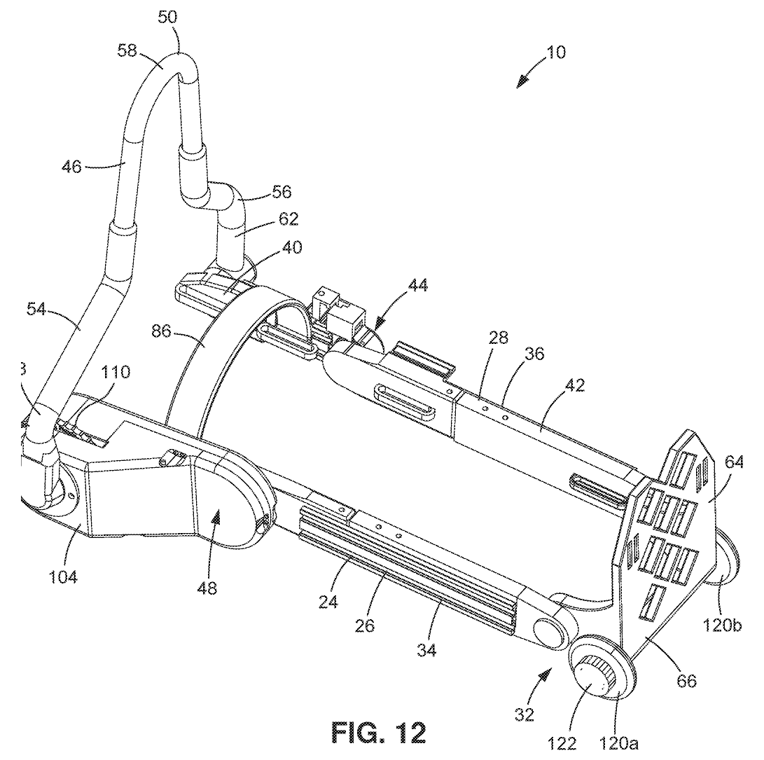

According to an aspect of the invention, with reference to FIGS. 1-8 and 12, there is provided an apparatus 10 for knee extension and flexion worn about a leg 14 of a user 12. The apparatus 10 includes a frame 24 defining a generally elongated rectangular-like frame having a generally parallel first and second frame sides 26, 28, a frame handle end 30 and a foot support end 32 bent towards the frame handle end 30. The first frame side 26 includes a first upper leg portion 34, a first lower leg portion 36, and a first knee hinge 38 disposed between and connecting the first upper leg portion 34 and the first lower leg portion 36. The first knee hinge 38 is sized and configured to allow the first lower leg portion 36 to rotate relative to the first upper leg portion 34. The second frame 28 side includes a second upper leg portion 40, a second lower leg portion 42, and a second knee hinge 44 disposed between and connecting the second upper leg portion 40 and the second lower leg portion 42. The second knee hinge 44 is sized and configured to allow the second lower leg portion 42 to rotate relative to the second upper leg portion 40. The apparatus 10 further includes a handle 46 having a handle pivot end 48 and a handle distal end 50. The handle pivot end 48 is connected to the frame handle end 30. The handle 46 is sized and configured to allow the handle 46 to pivot relative to the frame 24. The handle 46 is disposed in mechanical communication with the first lower leg portion 36 and sized and configured to rotate the first lower leg portion 36 relative to the first upper leg portion 34 upon pivoting of the handle 46 relative to the frame 24. The apparatus 10 further includes a knee angle sensor 52 sized and configured to measure an angle at the first knee hinge 38 between the first upper leg portion 34 and the first lower leg portion 36 and electronically generate angle data related to the measured angle. The knee angle sensor 52 is symbolically depicted in FIG. 1 with a box in dashed lining adjacent to the first knee hinge 38 and in the schematic portion of the drawing.

With this above described apparatus 10, the apparatus 10 may be worn about leg 14 of the user 12 with the first and second lower leg portions 36, 42 positioned adjacent to a lower leg 20 of the user 12, the first and second knee hinges 38, 44 adjacent to a knee 18 of the user 12, and the first and second upper leg portions 34, 40 positioned adjacent to the upper leg 16 of the user 12. With this arrangement, it is contemplated that movement of the first and second lower leg portions 36, 42 may in turn move the lower leg 20 of the user 12 to effect different angular positions of the lower leg 20 relative to the upper leg 16. In this respect the knee angle sensor 52 may be used to measure an angle of the lower leg 20 relative to the upper leg 16, assuming the frame 24 is securely engaged with the upper leg 16, the knee 18 and the lower leg 20 of the user 12. The apparatus 10 may be used for knee rehabilitation, such as for post-knee surgery, where the knee 18 is required to be put in extension/contraction and flexion. During knee rehabilitation, the user’s knee 18 may be only able to be extended/contract and flexed at certain limited angles. Over time during the rehabilitation using the apparatus 10 the range of angular motion for the knee 18 may be increased.

The apparatus may include various components to secure the apparatus 10 to the leg of the user 12. The apparatus 10 may include a thigh support 86. The thigh support 86 be a curved and relatively stiff member that spans between the first and second upper leg portions 34, 40. The thigh support 86 may be formed of a plastic or light weight metal with a curvature to receive a thigh of the user 12 when the apparatus is worn. The thigh support 86 is used to not only restrain the upper leg 16 but also to provide structural integrity of the overall frame 24. A thigh support strap 88 (indicated in dashed lining) and a top upper leg strap 90 (indicated in dashed lining) may be provided that are used to secure the upper leg 16 of the user 12 to the first and second upper leg portions 34, 40. A top lower leg strap 92 and a bottom lower leg strap 94 (indicated in dashed lining) may be additionally provided that are used to secure the lower leg 20 to the first and second lower leg portions 36, 42.

The apparatus 10 may include a foot support 64 disposed adjacent the foot support end 32 and spanning across the first and second frame sides 26, 28. The foot support 64 is sized and configured to engage a bottom of a foot 22 of the user 12 being disposed against the foot support 64. Further, a heel support 66 may be provided to engage a heel of the foot 22 to restrain the foot 22 from moving rearward relative the foot support 64 during use. The foot support 64 may include a foot strap 96 (indicated in dashed lining) that spans across a top of the foot 22 of the user 12 to secure the position of the foot 22 when the apparatus is worn.

In the embodiment depicted, the handle 46 is connected to the first upper leg portion 34 and the second upper leg portion 40 at the frame handle end 30. The handle 46 may include a handle first section 54, handle second section 56 and a handle main section 58 disposed between the handle first section 54 and the handle second section 56. The handle first section 54 may include a first section attachment end 60 and the handle second section 56 may include a second section attachment end 62. The handle 46 is connected to the frame handle end 30 with the first section attachment end 60 attached to the first upper leg portion 34 and the second section attachment end 62 attached to the second upper leg portion 40. As mentioned above, the handle 46 includes a handle pivot end 48 and a handle distal end 50. In this embodiment, the handle pivot end 48 coincides with the first and second section attachment ends 60, 62.

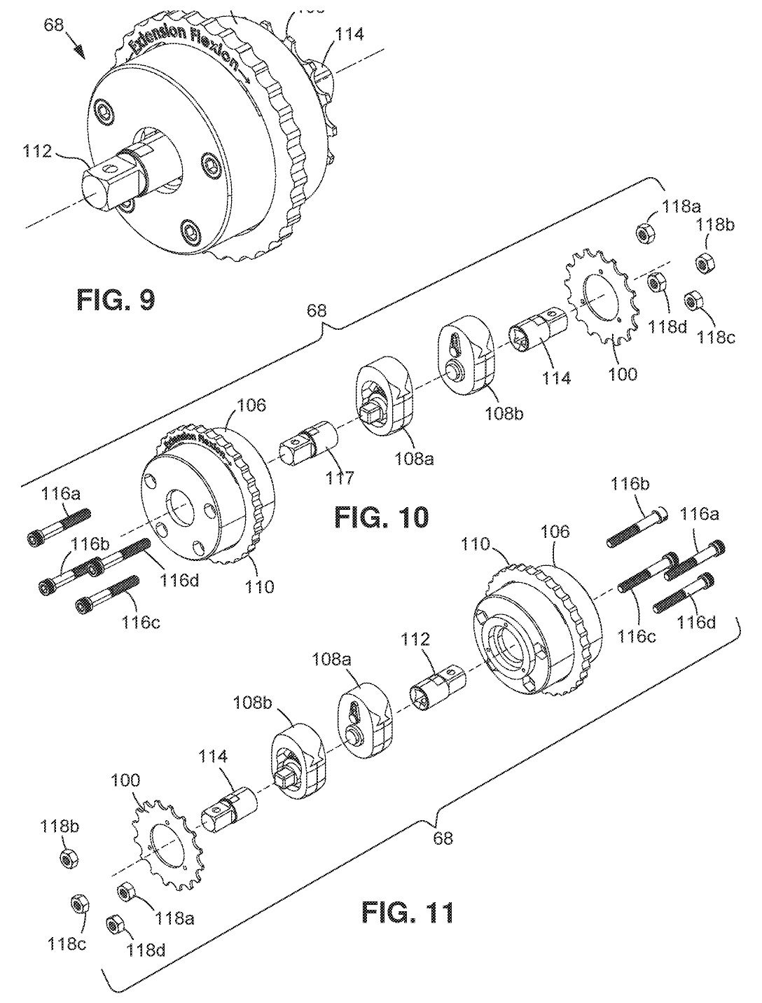

With additional reference to FIGS. 9-11, the apparatus 10 may include a ratchet mechanism 68 connected to the handle 46. FIG. 9 is a front perspective view of the ratchet mechanism 68, FIG. 10 is an exploded front perspective view of the ratchet mechanism 68 and FIG. 11 is an exploded back perspective view of the ratchet mechanism 68. The ratchet mechanism 68 is in mechanical communication with the first lower leg portion 36 and sized and configured to rotate the first lower leg portion 36 in a rotational direction relative to the first upper leg portion 34 upon pivoting of the handle 46 relative to the frame 24. In this embodiment, as discussed further below, the ratchet mechanism 68 is in mechanical communication with the first lower leg portion 36 through the use of a drive chain 102.

The ratchet mechanism 68 includes a ratchet housing 106 that contains nested cams 108a,b, a selection wheel 110, a handle extension 112 and a drive extension 114. Bolts 116a-d and nuts 118a-d are used to assemble the ratchet housing 106. A handle sprocket 100 is attached to the ratchet housing 106. The nested cams 108a,b, a selection wheel 110, a handle extension 112 and a drive extension 114 are aligned to rotate about a common axis with the handle sprocket 100. The handle first section 54 is attached to the handle extension 112 at the first section attachment end 60. A pivoting of the handle 46 about the first section attachment end 60 in turn rotates the handle extension 112. The ratchet housing 106 includes internal curvatures that interact with nested cams 108a,b and the selection wheel 110 is configured to interact with the nested cams 108a,b. In this respect the ratchet mechanism 68 operates similar to that of a ratchet socket wrench that selectively rotates prevents rotation in a particular rotational direction while allowing rotation in an opposition rotational direction. This allows for sequential pivoting of the handle 46 to incrementally advance rotation of the handle sprocket 100 in a desired rotational direction. Rotation of the selection wheel 110 is used to select a rotational direction or no rotational direction as a result of axial rotation of the handle extension 112 attached to the pivoting handle 46. Rather than a selection wheel 110, other arrangements may be implemented such as a knob or lever. The ratchet mechanism 68 is constructed according to those configurations which are well known to one of ordinary skill in the art.

A hinge sprocket 98 is attached to the first lower leg portion 36. The hinge sprocket 98 is aligned with a common axis of rotation with the first knee hinge 38. The drive chain 102 is looped about the handle sprocket 100 and the hinge sprocket 98. Rotation of handle sprocket 100 advances the drive chain 102 which in turn rotates of drive sprocket 98. In this regard, the ratchet mechanism 68 is configured to selectively control direction of rotation of the first lower leg portion 36 relative to the first upper leg portion 34 upon pivoting of the handle 46 relative to the frame 24. A cover 104 may be provided to enclose the ratchet mechanism 68, the handle sprocket 100, the hinge sprocket 98 and drive chain 102. It is contemplated that other arrangements may be selected from those which are well known to one of ordinary skill in the art to facilitate the handle 46 being disposed in mechanical communication with the first lower leg portion 36 and sized and configured to rotate the first lower leg portion 36 relative to the first upper leg portion 34 upon pivoting of the handle 46 relative to the frame 24. For example, instead of a sprocket and drive chain arrangement, a belt drive, a rope and pulley system or multiple interlocking gears may be used. Further, the handle pivot end 48 may directly engage at the first knee hinge 38 rather than at the distal end of the first upper leg portion 34. It is contemplated that gear ratios may be advantageously selected to provide ease of user use with respect to the amount of force required to pivot the handle 46.

Advantageously, the apparatus 10 as a single device may be used to impart forces upon the knee 18 for both extension and flexion contracture of the leg 14 of the user 12. Further, it is contemplated that the use of the handle 46 is conveniently accessible by the user 12 in a geometry that allows for a large degree of leverage by the user 12 to the handle 24 to initiate rotation of the first lower leg portion 36 relative to the first upper leg portion 34.

As mentioned above the apparatus 10 further includes a knee angle sensor 52 sized and configured to measure an angle at the first knee hinge 38 and electronically generate angle data related to the measured angle. In addition to the apparatus 10 being configured to impart forces upon the lower leg 20 to effect both extension and flexion of the leg 14, the apparatus 10 is able to generate angle data. The collection of angle data is critical in the assessment of progress and treatment of leg / knee therapy.

With reference to FIG. 1, the apparatus 10 may include electronics 70 that include a processor 72, a power source 74 and a transmitter 76 as symbolically depicted in the schematic portion of the drawing. The electronics 70 may include those circuit boards and electrical components which are well known to one of ordinary skill in the art. The electronics 70 may be attached to the first upper leg portion 34 and housed in the cover 104. The angle knee angle sensor 52 is in electrical communication with the processor 72. The processor 72 and the transmitter 76 are powered by the power source 74. The processor 72 is in electronic communication with the knee angle sensor 52 and sized and configured to receive the angle data. The transmitter 76 is in electronic communication with the processor and sized and configured to transmit a signal related to the angle data. In this respect a remote device 78 is symbolically depicted which may be configured to receive the transmitted signal related to the angle data. An “app” may be configured for the collection and display of angle data which is critical in the assessment of progress and treatment of leg / knee therapy. In this respect the apparatus 10 is contemplated to be a “smart” therapeutic device.

A wheel position input 84 make take the form of an electronic component configured to detect the position of the selection wheel to determine the selected direction of rotation or no rotation of the output handle sprocket 100 (which in turn controls the rotational direction of the hinge sprocket 98). Wheel position input 84 provides an electronic signal to the processor 72 which may be transmitted.

The angle sensor may take the form of any of those devices which are known to one of ordinary skill in the art to make angle measurements, which may include for examples optical encoders, magnetic encoders, absolute encoders and incremental encoders (uses pulses).

The apparatus 10 may further include a force sensor 80 that is sized and configured to sense torque at the knee hinge 38 with respect to forces applied to the first lower leg portion 36. The force sensor 80 is configured to electronically generate force data related to the sensed torque. The force sensor 80 symbolically depicted in FIG. 1 with a box in dashed lining adjacent to the first upper leg portion 34 and in the schematic portion of the drawing. The processor 72 is in electronic communication with the force sensor 80 and sized and configured to receive the force data. The force sensor 80 may take the form of strain gauges, force transducers, capacitor devices, optical devices, piezoelectric devices or any of those other devices which are well known to one of ordinary skill in the art. The transmitter 76 is in electronic communication with the processor 72 and sized and configured to transmit a signal related to the force data. The force sensor 72 may be sized and configured to sense torque at the first knee hinge 38 with respect to forces applied to the first lower leg portion 36 by sensing torque with respect to the ratchet mechanism 68. In particular, torque may be detected at the handle sprocket 100. As with the angle data, the collection of force data may be critical in the assessment of progress and treatment of leg / knee therapy.

Heel wheels 120a,b may be disposed adjacent the foot support sized and configured to rotate upon rolling of the heel wheels 120a,b along a surface 124 for movement of the foot support 64. It is contemplated that such rolling movement may be particularly useful when cycling the leg 14 through extension and flexion contractures in a controlled and smooth manner. The heel wheel 120a may be sized and configured to selectively change rolling resistance of the heel wheel 120a. In this respect, a simple resistance knob 122 may be integrated with the heel wheel 120a and configured to increase and decrease tension in heel wheel 120a upon rotation of the resistance knob 122 in either rotational direction. This may allow for selective resistance of movements of the leg 14 through extension and flexion contractures with the rolling movement. In another arrangement, resistance may be introduced adjacent the first knee hinge 38 rather than at the heel wheel 120a.

A resistance sensor 82 may be provided that is sized and configured to sense rolling resistance with respect to the heel wheel 120a. The resistance sensor 82 may electronically generate wheel resistance data related to the sensed rolling resistance. The resistance sensor is symbolically depicted in FIG. 1 with a box in dashed lining adjacent to the heel wheel 120a and in the schematic portion of the drawing. The processor 72 may be disposed in electronic communication with the resistance sensor 82 and sized and configured to receive the resistance data. The transmitter 67 may be sized and configured to transmit a signal related to the resistance data. As with the angle and force data, the collection of resistance data may be critical in the assessment of progress and treatment of leg / knee therapy.

The foot support 64 may be sized and configured to selectively change angular position relative to the first and second frame sides 26, 28. The ability to selectively change the angular position of the foot support 64 is contemplated to facilitate differing therapeutic treatments.

The electronics 70, the processor 72, the power source 74, the transmitter 76, the knee angle sensor 52, the force sensor 80, the resistance sensor 82 and the wheel position input 84 may be constructed, selected and configured according to any of those methods which are well known to one of ordinary skill in the art.

The frame 24 and the handle 46 may be formed of any of those materials, shaped and configured according to any of those which are well known to one of ordinary skill in the art. For example, the material may be of metal, like aluminum, or stiff plastic. The frame 24 and handle 46 may be formed of multiple components. The overall length of the frame 24 may be adjusted. In the embodiment depicted, first and second lower leg portions 36, 42 may be telescopically changed in length and selectively locked into place, such a via spring pin and hole arrangement.

The particulars shown herein are by way of example only for purposes of illustrative discussion, and are presented in the cause of providing what is believed to be the most useful and readily understood description of the principles and conceptual aspects of the various embodiments set forth in the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. An apparatus for knee extension and flexion worn about a leg of a user, the apparatus comprising:
a frame defining a generally elongated rectangular-like frame having a generally parallel first and second frame sides, a frame handle end and a foot support end bent towards the frame handle end, the first frame side including a first upper leg portion, a first lower leg portion, and a first knee hinge disposed between and connecting the first upper leg portion and the first lower leg portion, the first knee hinge being sized and configured to allow the first lower leg portion to rotate relative to the first upper leg portion, the second frame side including a second upper leg portion, a second lower leg portion, and a second knee hinge disposed between and connecting the second upper leg portion and the second lower leg portion, the second knee hinge being sized and configured to allow the second lower leg portion to rotate relative to the second upper leg portion;
a handle having a handle pivot end and a handle distal end, the handle pivot end connected to the frame handle end, the handle being sized and configured to allow the handle to pivot relative to the frame, the handle being disposed in mechanical communication with the first lower leg portion and sized and configured to rotate the first lower leg portion relative to the first upper leg portion upon pivoting of the handle relative to the frame; and
a knee angle sensor sized and configured to measure an angle at the first knee hinge between the first upper leg portion and the first lower leg portion and electronically generate angle data related to the measured angle.

2. The apparatus of claim 1 further includes a ratchet mechanism connected to the handle, the ratchet mechanism in mechanical communication with the first lower leg portion and sized and configured to rotate the first lower leg portion in a rotational direction relative to the first upper leg portion upon pivoting of the handle relative to the frame.

3. The apparatus of claim 2 wherein the ratchet mechanism is configured to selectively control direction of rotation of the first lower leg portion relative to the first upper leg portion upon pivoting of the handle relative to the frame.

4. The apparatus of claim 2 further includes a handle sprocket attached to the ratchet mechanism, a hinge sprocket disposed attached to the first lower leg portion adjacent the first knee hinge, and a drive chain engaged with the handle sprocket and the hinge sprocket, the drive chain being sized and configured to rotate the hinge sprocket upon rotation of the handle sprocket, rotation of the hinge sprocket rotates the first lower leg portion relative to the first upper leg portion.

5. The apparatus of claim 1 wherein the handle is connected to the first upper leg portion and the second upper leg portion at the frame handle end.

6. The apparatus of claim 1 further includes a processor in electronic communication with the knee angle sensor and sized and configured to receive the angle data, the apparatus further includes a transmitter in electronic communication with the processor and sized and configured to transmit a signal related to the angle data.

7. The apparatus of claim 1 further includes a force sensor sized and configured to sense torque at the knee hinge with respect to forces applied to the first lower leg portion and electronically generate force data related to the sensed torque.

8. The apparatus of claim 7 further includes a processor in electronic communication with the force sensor and sized and configured to receive the force data, the apparatus further includes a transmitter in electronic communication with the processor and sized and configured to transmit a signal related to the force data.

9. The apparatus of claim 7 further includes a ratchet mechanism connected to the handle, the ratchet mechanism in mechanical communication with the first lower leg portion and sized and configured to rotate the first lower leg portion in a rotational direction relative to the first upper leg portion upon pivoting of the handle relative to the frame.

10. The apparatus of claim 9 wherein the force sensor is sized and configured to sense torque at the first knee hinge with respect to forces applied to the first lower leg portion by sensing torque with respect to the ratchet mechanism.

11. The apparatus of claim 1 further includes a foot support disposed adjacent the foot support end and spanning across the first and second frame sides, the foot support is sized and configured to engage a bottom of a foot of the user being disposed against the foot support.

12. The apparatus of claim 11 further includes a heel wheel disposed adjacent the foot support sized and configured to rotate upon rolling of the heel wheel along a surface for movement of the foot support.

13. The apparatus of claim 12 wherein the foot support is sized and configured to selectively change angular position relative to the first and second frame sides.

14. The apparatus of claim 12 wherein the heel wheel is sized and configured to selectively change rolling resistance of the heel wheel.

15. The apparatus of claim 14 further includes a resistance sensor sized and configured to sense rolling resistance with respect to the heel wheel and electronically generate wheel resistance data related to the sensed rolling resistance.

16. The apparatus of claim 15 further includes a processor in electronic communication with the resistance sensor and sized and configured to receive the resistance data, the apparatus further includes a transmitter in electronic communication with the processor and sized and configured to transmit a signal related to the resistance data.

17. An apparatus for knee extension and flexion worn about a leg of a user, the apparatus comprising:
a frame defining a generally elongated rectangular-like frame having a generally parallel first and second frame sides, a frame handle end and a foot support end bent towards the frame handle end, the first frame side including a first upper leg portion, a first lower leg portion, and a first knee hinge disposed between and connecting the first upper leg portion and the first lower leg portion, the first knee hinge being sized and configured to allow the first lower leg portion to rotate relative to the first upper leg portion, the second frame side including a second upper leg portion, a second lower leg portion, and a second knee hinge disposed between and connecting the second upper leg portion and the second lower leg portion, the second knee hinge being sized and configured to allow the second lower leg portion to rotate relative to the second upper leg portion;
a handle having a handle pivot end and a handle distal end, the handle pivot end connected to the frame handle end, the handle being sized and configured to allow the handle to pivot relative to the frame, the handle being disposed in mechanical communication with the first lower leg portion and sized and configured to rotate the first lower leg portion relative to the first upper leg portion upon pivoting of the handle relative to the frame; and
a force sensor sized and configured to sense torque at the knee hinge with respect to forces applied to the first lower leg portion and electronically generate force data related to the sensed torque.

18. The apparatus of claim 17 further includes a processor in electronic communication with the force sensor and sized and configured to receive the force data, the apparatus further includes a transmitter in electronic communication with the processor and sized and configured to transmit a signal related to the force data.

19. The apparatus of claim 17 further includes a ratchet mechanism connected to the handle, the ratchet mechanism in mechanical communication with the first lower leg portion and sized and configured to rotate the first lower leg portion in a rotational direction relative to the first upper leg portion upon pivoting of the handle relative to the frame.

20. The apparatus of claim 19 wherein the force sensor is sized and configured to sense torque at the first knee hinge with respect to forces applied to the first lower leg portion by sensing torque with respect to the ratchet mechanism.

21. The apparatus of claim 17 further includes a knee angle sensor sized and configured to measure an angle at the first knee hinge between the first upper leg portion and the first lower leg portion and electronically generate angle data related to the measured angle.
